(19) United States
(12) Patent Application Publication
(10) Pub. No.: US 2022/0370980 A1
(43) Pub. Date: Nov. 24, 2022

(54) COMPOSITE MATERIALS COMPRISING CONCRETE AGGREGATES, AND POROUS CARBON AND USE THEREOF FOR ELIMINATING POLLUTANT GASES

(71) Applicants: Centre National de la Recherche Scientifique, Paris (FR); École Spéciale des Travaux Publics, du Bâtiment et de I'Industrie, Cachan (FR)

(72) Inventors: Encarnacion RAYMUNDO-PIÑERO, SAINT-CYR-EN-VAL (FR); Jena JEONG, ORLÉANS (FR); Rachelle OMNÉE, SOINGS-EN-SOLOGNE (FR)

(21) Appl. No.: 17/755,136

(22) PCT Filed: Oct. 20, 2020
(86) PCT No.: PCT/FR2020/051883

§ 371 Date: Apr. 21, 2022

(30) Foreign Application Priority Data
Oct. 22, 2019 (FR) FR1911794

(51) Int. Cl. B01J 20/20; C04B 28/02; C04B 18/16; C04B 14/02; B01J 20/16; B01J 20/28; B01J 20/30; A61L 9/014; B01D 53/04

(52) U.S. Cl. CPC B01J 20/20; C04B 28/02; C04B 18/167; C04B 14/022; B01J 20/16; B01J 20/2803; B01J 20/28064; B01J 20/28066; B01J 20/28004; B01J 20/28071; B01J 20/3042; A61L 9/014; B01D 53/04; C04B 2103/0078

(57) ABSTRACT

The invention belongs to the field of eliminating pollutant gases. In particular, the invention belongs to the field of pollutant gas-absorbing material such as CO2, SO2, NOx and VOCs.

The present invention relates to a fresh composite or composite paste and a composite material comprising aggregates of recycled concrete, porous carbon, a binder and optionally water, as well as to the method for manufacturing the composite and the use thereof for sanitizing air (indoor or outdoor). The invention also relates to an article (for example, an anti-noise wall, a tunnel lining, an indoor decoration, an item of street furniture, etc.) comprising the composite according to the invention.

The invention belongs to the field of eliminating pollutant gases. In particular, the invention belongs to the field of pollutant gas-absorbing materials such as CO₂, SO₂, NOₓ and VOCs (volatile organic compounds).

The present invention relates to a fresh composite or composite paste and a composite material comprising aggregates of recycled concrete, porous carbon, a binder and optionally water, as well as to the method for manufacturing the composite and the use for sanitizing air (indoor or outdoor). The invention also relates to an article (for example, an anti-noise wall, a tunnel lining, an indoor decoration, an item of street furniture, etc. . . . ) comprising the composite according to the invention.

Global warming, also called planetary warming or climate disruption, is the phenomenon of increasing average oceanic and atmospheric temperatures. The causes of this global warming are now known and it is mainly due to greenhouse gas (GHG) emissions. Global warming refers mainly to the worldwide warming observed since the beginning of the 20th century.

The Intergovernmental Panel on Climate Change (IPCC) created in 1988 by the UN to synthesize scientific studies on climate concluded that “most of the observed increase in global average temperature since the mid-20th century is very likely attributable to rising anthropogenic GHG concentrations” (Climate Change 2007, Synthesis Report, IPCC). The 2014 report states, “Anthropogenic greenhouse gas emissions, which have increased since pre-industrial times largely due to economic and population growth, are currently higher than ever before, resulting in atmospheric concentrations of carbon dioxide, methane, and nitrous oxide that are unprecedented in at least 800,000 years. Their effects, along with those of other anthropogenic factors, have been detected throughout the climate system and are extremely likely to have been the primary cause of the observed warming since the mid-20th century.”

The latest IPCC projections are that the global surface temperature could rise by an additional 1.1 to 6.4° C. over the 21st century. The differences between projections come from different model sensitivities for greenhouse gas concentrations and different future emissions scenarios. Most studies have chosen 2100 as the time horizon, but warming is expected to continue beyond that because, even if emissions stopped, the oceans have already stored a lot of heat, carbon sinks need to be restored, and the lifetime of carbon dioxide and other greenhouse gases in the atmosphere is long.

The approach generally used to reduce pollutant gases in the atmosphere, such as CO₂, is to act directly on the sources of CO₂. Countries have decided to mobilize each year through Conferences of the Parties. At the COP21 in Paris, 149 countries had agreed to the same objective of fighting against greenhouse gases. Since the Rio agreement and the Kyoto protocol, GHG emissions (especially CO₂) have decreased slightly but these will not be enough to keep the atmosphere healthy and to slow down the increase of the average temperature observed for some years on the surface of the Earth. The European law (Euro6 standard) controlling the emission of gas from vehicles is becoming increasingly strict and the thermal regulations in the building industry are being redefined continuously (RT2015 and soon RT2020). The measures taken are therefore the application of new regulations such as the CO₂ tax to industrial companies such as cement factories and oil companies in order to limit the release of pollutant gases into the atmosphere.

Unfortunately, these efforts are still insufficient. The proposal to act directly on the sources of GHGs (especially CO₂, as for example the substitution method on cement clinker) exists in many fields, but the various studies carried out show that there is also an urgent need to act on the CO₂ already present in the atmosphere.

To date, the amount of CO₂ in the atmosphere is far too high. Furthermore, eliminating pollutant gases in urban areas has never been satisfactorily solved, not least because of recurring economic and marketing problems.

It is therefore essential, in addition to fighting the causes of greenhouse gas emissions, to eliminate and store the pollutant gases already present in the atmosphere.

In a perspective of multi-benefit between the revalorization of waste via re-carbonation and the reduction of CO₂ gas responsible for the greenhouse effect, projects have been launched around the world. The objective of these projects is on the one hand to reduce the waste of building industry and to recycle them but also to capture the CO₂ through the aggregates of recycled concrete in the double purpose to decrease the quantity of CO₂ in the atmosphere and to improve the quality of the chemical-physical properties of the recycled concrete. Indeed, to manufacture cement clinker, the “raw material” of cement, the firing of limestone and clay in kilns at very high temperatures, releases large quantities of CO₂. This decarbonation stage represents about 60% of the CO₂ emissions per ton produced (equivalent to about 700 kg of CO₂ released per ton of cement produced!). However, the world cement production represents more than 3 billion tons per year, of which more than 16 thousand tons come from the 12 cement plants in France). Consequently, these studies aim at recovering essentially the smoke coming out of the cement plant chimneys by the aggregates of recycled concrete in order to store the CO₂ in the recycled concrete by the phenomenon of accelerated carbonation. In order to optimize these procedures, the CO₂ recovery must be fast while ensuring the total carbonation of the recycled concrete because this latter recovers and traps a maximum of CO₂.

It is noted that according to theoretical analysis (Steinour H H, Journal of American Concrete Institute, 1956, 30:905-907), the maximum CO₂ captured by cement would be limited to about 50% mass (equivalent to 500 kg of CO₂ per ton of cement) because of the limited amount of chemical elements in cement.

However, these quantities are still insufficient and there is no material, which can be designed from recycled material, capable of absorbing a higher quantity of CO₂, and even of absorbing other pollutant gases such as SO₂, NOₓ and VOCs.

Moreover, today even if recycled concrete is used for road works and fills mainly, this application does not cover all the waste and landfills are still a widely practiced solution now.

It is also noted that in confined or poorly ventilated atmospheres, such as classrooms or certain indoor work environments, the concentration of CO₂ can be up to 10 times higher than in an open environment.

There is therefore a need to develop composite materials that overcome the drawbacks described in the state of the art. In particular, there is a need for a composite that significantly increases both the absorption rate and the amount of absorbed pollutants.

To the credit of the applicant, a new type of composite material has been developed comprising aggregates of recycled and porous carbon. The combination of these two elements synergistically improves the absorption properties of each of these elements taken separately. The amount of CO₂ absorbed can go beyond 15% mass percent (based on the total mass of the composite material), at least 5% more than known methods which generally cap at 10% (for example crushed concrete aggregate).

At this time, to the best of the applicant's knowledge, there is currently no material, let alone a composite material of any kind, that meets these requirements, both in terms of absorption rate and the amount of pollutant gases absorbed.

The present invention thus relates first to a new fresh composite or composite paste and a new composite material, comprising aggregates of recycled concrete and porous carbon. The method for manufacturing this new material is furthermore simple and inexpensive. They present improved performances compared to existing absorbent materials, in particular in terms of absorption efficiency (rate and quantity of absorbed gases).

The invention thus relates to a fresh composite or composite paste comprising at least one concrete aggregate having a porosity greater than or equal to 12%, porous carbon having a micropore volume greater than or equal to 0.2 cm/g³ and a macropore volume greater than or equal to 0.2 cm/g³, a binder and optionally water.

Advantageously, the fresh composite according to the invention may comprise at least one concrete aggregate having a porosity greater than or equal to 12%, porous carbon having a micropore volume greater than or equal to 0.2 cm/g³ and a macropore volume greater than or equal to 0.2 cm/g³, a binder and water.

In the present application, “fresh composite” or “composite paste” means the heterogeneous paste formed by mixing concrete aggregates, porous carbon, binder, optionally water and optionally additives, adjuvants and/or aggregates, prior to curing leading to the composite according to the invention.

“Concrete aggregate” means concrete residues that may be recycled concrete that has been crushed or ground into aggregate. It can also contain residues such as brick, ceramic, glass and other elements (mainly those with a density close to that of concrete) that can be found in some concrete. According to the article, “Porosity of recycled concrete with substitution of aggregate of recycled concrete—An experimental study” (Cement and Concrete Research, 32, 2002, 1301-1311), the porosity of aggregate of recycled concrete is generally between 13% and 15%.

In the context of the invention, “porosity” means all the voids (pores or spaces) in a solid material. The pores may be filled by fluids such as liquids or gases. The porosity φ of a porous medium A can also be represented by a numerical value defined as the ratio of the total pore volume (Vₚₒᵣₑₛ) to the total volume of the porous medium (Vₜₒₜₐₗ): φ_{A}=Vₚₒᵣₑₛ/Vₜₒₜₐₗ.

0<φ_{A}<1

This defines φ_{concrete aggregate} and φ_{porous carbon}. The pores can be intra- or interparticle. Both the concrete aggregate and the porous carbon possess intraparticle spaces. With respect to porous carbon there are additionally interparticle spaces which are generally due to the spaces between the graphene sheets that make up said porous carbon.

Advantageously, the concrete aggregate may have a porosity greater than or equal to 12%, φ_{concrete aggregate}≤0.12.

Advantageously, the concrete aggregate may consist essentially of recycled concrete. The recycled concrete may include residues or chips of brick, ceramic, glass and other elements that may be found in some concrete.

Advantageously, the concrete aggregate can have an average diameter ranging from 1 to 50 mm, preferably from 1 to 20 and even more preferably from 5 to 10 mm. The size of the concrete aggregate may vary depending on the intended use of the composite material. Depending on the surface area of the concrete accessible to the gas, smaller diameter aggregates are generally carbonated faster and saturate faster in the short term than larger aggregates because the surface area accessible to the gas is larger than for the latter. Larger diameter aggregates will carbonate more slowly. These different kinetics can be taken into account according to the different uses of composite materials according to the invention that the person skilled in the art envisages.

Advantageously, the fresh composite according to the invention may comprise from 25 to 45% by weight of concrete aggregates, preferably 30 to 40%.

In the present application, “porous carbon” means residues of waste biomass, polymer, mineral carbons or residues of petroleum processes. The porous carbons can thus be obtained by thermal decomposition (pyrolysis) of a precursor such as biomass, polymer, mineral carbons or residues of petroleum processes. After pyrolysis, the porosity is generated by a chemical reaction at high temperature with an “activating agent” which can be selected from the group comprising CO₂, H₂O, KOH, NaOH and H₂PO₄. In the context of the invention, algae (biomass) are preferred as a precursor because it has been shown in previous work that with a single pyrolysis step, porous carbon materials can be obtained, thanks to the elements present in the algae composition. This is also the case with biomass waste rich in cellulose (E. Raymundo-Piñero et al., Advanced Materials, 2006, 18, 1877-1882).

Advantageously, the porous carbon can be composed essentially of carbon. It can have a more or less ordered structure with a large specific surface and a high degree of porosity. The porous carbon can be in the form of powder (with particle sizes ranging from μm to mm) or pellets in the composite according to the invention. By pellet is meant an aggregate resulting from a mixture of a carbon powder and a binder. Preferably, this binder can be a carbon such as petroleum pitch or carbon.

Advantageously, the porous carbon can have a micropore volume greater than or equal to 0.2 cm³/g, preferably greater than or equal to 0.0 4cm³/g. By “micropore” is meant a pore with a diameter of less than 2 nm.

Advantageously, the porous carbon can have a mesopore volume greater than or equal to 0.2 cm³/g, preferably ranging from 0.2 to 0.6 cm³/g. By “mesopore” is meant a pore with a diameter ranging from 2 to 50 nm. They are generally not involved in absorption, but can be useful in CO₂ transport.

Advantageously, the porous carbon can have a macropore volume greater than or equal to 0.2cm³/g, preferably greater than or equal to 1 cm³/g. By “macropore” is meant a pore with a diameter greater than 50 nm.

Advantageously, the powders, particles or aggregates of porous carbon can have an average diameter ranging from 1 to 20 mm, preferably from 3 to 15 and even more preferably from 5 to 10 mm. The size of the particles or aggregates of porous carbon can vary according to the use of the composite material.

Advantageously, the porous carbon may be functionalized, preferably on the surface. The surface functionalization may be of at least one heteroatom selected from the group comprising O, N, S and P.

Advantageously, the porous carbon can have a specific surface area ranging from 500 to 3000 m²/g, preferably from 700 to 2500 m²/g, and even more preferably from 1500 to 2500 m²/g.

Advantageously, the fresh composite according to the invention may comprise from 1 to 20% by weight of porous carbon, preferably 1 to 10%.

In this application, “binder” means a finely ground material that reacts with water to form a paste that sets and hardens after mixing with water. This can be cement, but also polymers, resins or carbon-based materials (pitch). Advantageously, when the binder is a polymer, resin or carbon-based material (pitch), the fresh composite and the composite according to the invention generally do not contain water.

Advantageously, the binder can be selected from the group comprising cements, preferably cements of category CEM I (Portland cement), CEM II A or B (blended Portland cement), CEM III A, B or C (blast furnace cement), CEM IV A or B (pozzolanic type cement) or CEM V A or B (blended cement).

Advantageously, the binder can be selected from the group comprising polymeric or copolymeric binders, preferably binders of the unsaturated polyester type, epoxy, acrylic or vinyl acetate resins, vinyl esters, phenolic resins, polyurethane resins, polyethylene, polystyrene, polycarbonate, latex, alkyl copolymers, epoxy, acrylonitrile, polyvinyl chloride, polyurethane, chlorinated polymeric binders, and their mixtures.

Advantageously, the binder can be selected from the group comprising carbon-based binders, preferably carbon pitch or petroleum pitch type binders.

Advantageously, the fresh composite according to the invention may comprise from 20 to 60% by weight of binder, preferably 30 to 50%.

Advantageously, the fresh composite according to the invention may comprise from 10 to 25% by weight of water, preferably 15 to 20%.

Advantageously, the fresh composite according to the invention may comprise:
from 25 to 45% by weight of at least one concrete aggregate having a porosity greater than or equal to 12%,
from 1 to 20% by weight of porous carbon having a micropore volume greater than or equal to 0.2 cm³/g and a macropore volume greater than or equal to 0.2 cm³/g,
from 20 to 60% by weight of a binder, and
optionally from 10 to 25% by weight of water.

Advantageously, the fresh composite according to the invention may have a volume ratio of concrete aggregate/porous carbon in the range of from 30:70 to 80:20, preferably from 40:60 to 60:40.

Advantageously, the fresh composite according to the invention may have a ratio of concrete aggregate outer surface/porous carbon outer surface in the range of 0.5 to 1.5.

Advantageously, the fresh composite according to the invention may have a mass ratio of concrete aggregate/binder in the range of 0.6 to 1.

Advantageously, the fresh composite according to the invention may have a mass ratio of porous carbon/binder in the range of 0.03 to 0.1.

Advantageously, the fresh composite according to the invention may have a mass ratio of concrete aggregate/(binder+water) ranging from 0.4 to 0.8.

Advantageously, the fresh composite according to the invention may have a mass ratio of porous carbon/(binder+water) ranging from 0.02 to 0.08.

Advantageously, the fresh composite according to the invention may further comprise at least one adjuvant and/or additive.

Advantageously, the fresh composite according to the invention may comprise from 0 to 2% of at least one adjuvant and/or additive.

“Adjuvant” or “additive” means chemical products usually added during the mixing of concrete and slightly dosed during the preparation (less than 5% of the mass of the concrete). These products offer the possibility of improving certain characteristics of the concrete such as its setting time or its waterproofing. In the context of the invention, adjuvants and/or additives can be incorporated during the manufacture of the concrete, more particularly during the mixing of the binder with the concrete aggregates and the porous carbon, in order to improve its properties. An adjuvant can have an effect on several parameters: strength, fluidity, setting time and permeability of the composite according to the invention.
Fluidity: this is generally provided by plasticizers and super-plasticizers. These products also increase the strength of the composite in the cured state.
Setting time: this can generally be regulated by incorporating an accelerator or a setting retarder.
Permeability: this can usually be increased by incorporating an air entrainer, which creates micro-bubbles in the composite. Conversely, mass water repellent limits the penetration of water into the pores and capillaries of the composite.
Additives are generally considered as adjuvants but have a more specific role of lowering the shear threshold to modify the rheological behavior of fresh concrete. They can be, for example, superplasticizers.

Advantageously, the fresh composite according to the invention may comprise one or more adjuvants and/or additives selected from the group comprising setting accelerators, curing accelerators, setting retarders, plasticizers, water-reducing plasticizers, superplasticizers (fluidizing or reducing), air entrainers, water repellents, pigments or dyes, and curing agents.

Advantageously, the composite according to the invention may further comprise at least one aggregate.

“Aggregate” is a compound of mineral grains of varying sizes and shapes, the most common of which are called “sand” and “gravel”. They are generally used in the composition of concrete and mortar.

Advantageously, the fresh composite according to the invention can include silica fume, carbon nanotubes (to add mechanical strength), polymers (as superplasticizers to facilitate manufacturing and installation), dye-stuffs (for aesthetic appearance, expanded shale, polystyrene beads or even vegetable fibers.

Advantageously, the fresh composite according to the invention may comprise:
from 25 to 45% by weight of at least one concrete aggregate having a porosity greater than or equal to 12%,
from 1 to 20% by weight of porous carbon having a micropore volume greater than or equal to 0.2 cm³/g and a macropore volume greater than or equal to 0.2 cm³/g,
from 20 to 60% by weight of a binder and
optionally from 10 to 25% by weight of water.
optionally from 0 to 2% by weight of at least one adjuvant and/or additive.

The invention also relates to a new composite material obtained by curing the fresh composite according to the invention. The method for preparing these new materials is also simple and inexpensive. They have improved performance compared to existing absorbent materials, in particular in terms of absorption efficiency (rate and quantity of absorbed gases).

Thus, the invention also relates to a composite comprising at least one concrete aggregate having a porosity greater than or equal to 12%, porous carbon having a micropore volume greater than or equal to 0.2 cm³/g and a macropore volume greater than or equal to 0.2 cm³/g, a binder and optionally water.

Advantageously, the composite may comprise at least one concrete aggregate having a porosity greater than or equal to 12%, porous carbon having a micropore volume greater than or equal to 0.2 cm³/g and a macropore volume greater than or equal to 0.2 cm³/g, a binder and water.

Advantageously, the composite according to the invention may comprise from 25 to 45% by weight of concrete aggregates, preferably 30 to 40%.

Advantageously, the composite according to the invention may comprise from 1 to 20% by weight of porous carbon, preferably 1 to 10%.

Advantageously, the composite according to the invention may comprise from 20 to 60% by weight of binder, preferably 30 to 50%.

Advantageously, the composite according to the invention may comprise from 10 to 25% by weight of water, preferably 15 to 20%.

Advantageously, the fresh composite and the composite according to the invention have the same composition. In particular, the composite according to the invention generally comprises as much water as the fresh composite, the curing not being a drying but a crystallization. One can also speak of “semi-crystallization” in the sense that, in the paste, the CSH (or CSH gel) may be poorly crystallized and have sheet or flake shapes, whereas the Ca(OH)₂ is generally completely crystallized. With time, the quantity of water can nevertheless decrease: in particular when some water molecules have not been in contact with the dry cement, the mixing of the water with the dry cement not having been perfectly homogeneous. These water molecules can remain trapped and may evaporate with time. However, this quantity is generally small, and the composite according to the invention can have a quantity of water up to only 10% lower, compared to the quantity of water in the fresh composite according to the invention.

Advantageously, the composite according to the invention may have a volume ratio of concrete aggregate/porous carbon in the range of from 30:70 to 80:20, preferably from 40:60 to 60:40.

Advantageously, the fresh composite according to the invention may have a ratio of concrete aggregate outer surface/porous carbon outer surface in the range of 0.5 to 1.5.

Advantageously, the composite according to the invention may have a mass ratio of concrete aggregate/binder in the range of 0.6 to 1.

Advantageously, the composite according to the invention may have a mass ratio of porous carbon/binder in the range of 0.03 to 0.1.

Advantageously, the composite according to the invention may have a mass ratio of concrete aggregate/(binder+water) ranging from 0.4 to 0.8.

Advantageously, the composite according to the invention may have a mass ratio of porous carbon/(binder+water) ranging from 0.02 to 0.08.

Advantageously, the composite according to the invention may further comprise at least one adjuvant and/or additive, preferably in an amount less than or equal to 2%.

Advantageously, the composite according to the invention may comprise:
from 25 to 45% by weight of at least one concrete aggregate having a porosity greater than or equal to 12%,
from 1 to 20% by weight of porous carbon having a micropore volume greater than or equal to 0.2 cm/g³ and a macropore volume greater than or equal to 0.2 cm³/g,
from 20 to 60% by weight of a binder, and
optionally from 10 to 25% by weight of water.

Advantageously, the composite according to the invention may comprise:
from 25 to 45% by weight of at least one concrete aggregate having a porosity greater than or equal to 12%,
from 1 to 20% by weight of porous carbon having a micropore volume greater than or equal to 0.2 cm³/g and a macropore volume greater than or equal to 0.2 cm³/g,
from 20 to 60% by weight of a binder and
optionally from 10 to 25% by weight of water.
optionally from 0 to 2% by weight of at least one adjuvant and/or additive.

The invention also relates to a use of a composite according to the invention to sanitize air. The air may be air in an indoor or outdoor environment.

Advantageously, the use of the composite according to the invention may be to absorb pollutant gases included in the air. The composite according to the invention can thus be used to absorb CO₂, SO₂, NOx and VOCs.

“NOx” refers to nitrogen oxides, which are chemical compounds composed of oxygen and nitrogen.

“VOCs” or “volatile organic compounds” means organic compounds that can easily be found in gaseous form in the earth's atmosphere. They constitute a very broad family of products. These compounds have the particularity to have a very low boiling point, they evaporate or sublimate easily from their solid or liquid form. This gives them the ability to spread more or less far from their place of emission, thus causing direct and indirect impacts on animals and nature. VOCs can be of anthropic origin (from refining, evaporation of organic solvents, unburned, etc.) or of biotic origin (BVOCs or biogenic VOCs emitted by plants or certain fermentations). They can be biodegradable or not.

The invention also relates to a method for manufacturing a composite according to the invention.

Advantageously, the method for manufacturing a composite according to the invention may comprise the steps of:
a) mixing at least one concrete aggregate having a porosity greater than or equal to 12%, porous carbon having a micropore volume greater than or equal to 0.2 cm³/g and a macropore volume greater than or equal to 0.2 cm³/g, a binder, optionally water and optionally additives, adjuvants and/or aggregates, and obtaining a fresh composite;
b) molding and curing the fresh composite obtained in step a);
c) demolding.

The term “curing” or “hydration” means, particularly in the field of civil engineering, the hydration method that produces the more or less crystallized chemical elements of the final product, the two terms are generally used interchangeably because curing is the result of hydration (increase in the Young's Modulus value). When the fresh composite does not comprise water, the curing in step b) may be the chemical reaction of the polymeric binder, resin or carbon-based material to obtain the composite according to the invention.

Advantageously, the amounts of concrete aggregate, porous carbon, binder, water and optionally additive, adjuvant and/or aggregate in the mixture of step a) are as defined above.

Advantageously, step a) further comprises the addition of any adjuvant, additive and/or aggregate. Preferably, step a) can comprise the sub-steps:
i) mixing of concrete aggregates, binder, porous carbon and possible aggregate,
ii) homogenization, and
iii) optionally addition of water optionally comprising at least one adjuvant and/or additive, preferably in a progressive manner.

Advantageously, the curing step b) can be carried out in molds of desired dimensions. The person skilled in the art will know how to adapt the mold according to the desired final use of the composite.

Advantageously, step b) can last from 4 to 48 hours, preferably 24 hours. Preferably, the demolding step c) should be carried out less than 24 hours after the start of curing. This allows curing to continue after step c). The curing time can be shortened or lengthened depending on the adjuvants and/or additives that may be present in the fresh composite. The person skilled in the art will know how to adapt the duration of step b) according to the fresh composite mixture prepared.

Advantageously, the curing can have a total duration of at least 10 to 30 days, preferably at least 20 days and even more preferably at least 27 days.

Advantageously, the method according to the invention can be carried out at a temperature ranging from 10 to 35° C. At higher temperatures, water losses may occur. At lower temperatures, curing may be slowed.

The composite material according to the invention also has the following advantages:
it has a high added value because it can be prepared from waste materials (recycled concrete and biomass) and give them a new function as a depolluting material;
it has exceptional depolluting capacities by the combined presence of concrete aggregates and porous carbon;
it has the capacity to eliminate gases such as SO₂ and NOₓ by transforming them into non harmful products;
it can be manufactured simply with traditional techniques in prefabrication plants of cementitious materials to obtain products adapted for new works or to cover existing works;
the porous carbon included in the composite according to the invention is derived from vegetable waste of biomass, usually intended to provide energy by combustion, in which they are less valorized than in the invention and can even entail risks of overexploitation of the resources.

### BRIEF DESCRIPTION OF THE FIGURES

FIG. 1 represents a schematic of the composite manufacturing method according to the invention.

FIG. 2 shows the CO₂ capture capacity for composites 1 and 2. (a) CO₂ capture capacity as a function of composite material mass; (b) CO₂ capture capacity as a function of material volume; (c) CO₂ capture capacity as a function of gas accessible surface area.

FIG. 3 shows the diffusion of CO₂ into the cementitious material matrix in composite 2 (a) and composite 1 (b) after 20 days in a 10,000 ppm CO₂ atmosphere. The darker gray areas correspond to the non-carbonated cementitious material.

The invention is further illustrated by the following non-limiting examples.

Example 1: Synthesis of a composite according to the invention.

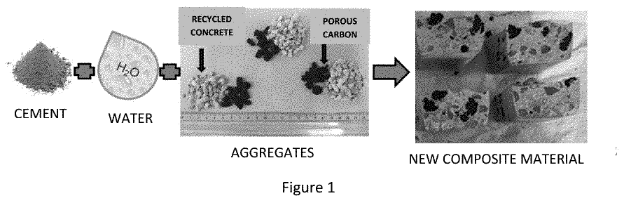

The composite material consists of aggregates of recycled concrete and porous carbon embedded in a cement matrix. The preparation method of the composites is like the classical method of concrete preparation (see FIG. 1). The different steps of the synthesis are:
1) Prepare the porous carbon by pyrolysis of brown algae (Lessonia nigrescens) in an oven at 750° C. and an atmosphere of 100 ml/min of inert gas (N₂),
2) Mix the aggregates of recycled concrete and the porous carbon with a volume ratio of recycled concrete : porous carbon of 60:40. For this purpose 319 g of aggregate of recycled concrete (13% porosity) with an average diameter of about 6 mm are mixed with 11.9 g of porous carbon particles with an average diameter of about 6 mm and a micropore volume of 0,4cm³/g and a macropore volume of 4,8cm³/g (specific surface area 746 m²/g).
3) Prepare the binder by making a cement paste with cement and water. The quantity of cement corresponds to a mass ratio aggregate/cement of about 0.8. The quantity of water corresponds to a W/C ratio of 0.45. For this purpose, 400 g of CEM I cement are mixed manually with 180 g of water.
4) Mix the aggregates with the binder by hand until the aggregates are homogeneously distributed in the “cement” paste.
   The result is fresh composite 1,
To prepare the composite, we continue with the steps:
4) Pour the fresh composite into a mold with dimensions 9*3*2 cm and let it harden for 24 hours in a chamber with a relative humidity of about 100%.
5) Demold and proceed to a hydration stage of 27 days in a climatic chamber with a relative humidity of about 100%.
   FIG. 1 in the appendix of a cross section of the composite materials shows that the distribution of aggregates in the cement matrix is homogeneous, although they are small pieces (3×3×2 cm) made at laboratory scale with manual mixing.

Example 2: Synthesis of a composite outside the invention.

The composite material consists of aggregates of recycled concrete embedded in a cement matrix. The preparation method of the composites is like the classical method of concrete preparation (see FIG. 1). The different steps of the synthesis are:
1) Prepare 474 g of aggregates of recycled concrete (13% porosity) with an average diameter of about 6 mm.
2) Prepare the binder by making a cement paste with cement and water. The quantity of cement corresponds to a mass ratio of aggregates/cement of about 1.2. The quantity of water corresponds to a W/C ratio of 0.45. For this purpose, 400 g of CEM I cement are mixed manually with 180 g of water.
3) Mix the aggregates with the binder manually until the aggregates are homogeneously distributed in the “cement” paste.
   The fresh composite 2 is obtained (out of the invention).
To prepare the composite the following steps are added:
4) Pour the fresh composite into a mold with dimensions 9*3*2 cm and let it harden for 24 hours in a chamber with a relative humidity of about 100%,
5) Demold and proceed to a hydration stage of 27 days in a climatic chamber with a relative humidity of about 100%.

TABLE 1
Summary of characteristics of composites 1 and 2.

| Example 1 (composite 1 according | Example 2 (composite 2 out of |
|---|---|
| to the invention) | invention) |
| 319 g of recycled concrete (247 | 474 g of recycled concrete (370 |
| mL) | mL) |
| 11.8 g of porous carbon LN750 | n/a |
| (124 mL) | |
| 400 g of CEM I cement (385 mL) | 400 g of CEM I cement (385 mL) |
| 180 g water (W/C ratio = 0.45) | 180 g water (W/C ratio = 0.45) |

Example 3: Measurement of the depolluting capacities of composites 1 and 2.

The depolluting capacities of the materials were determined on a laboratory scale with CO₂ capture experiments under so-called “accelerated” conditions.

For this purpose, the composite materials were placed in a CO₂ incubator conditioned in a relative humidity of about 63%, an ambient temperature of about 25° C. and a CO₂ concentration with an order of magnitude higher than what can be found in highly polluted urban areas. The concentration of CO₂ in the air of a large city like Paris is about 400-450 ppm while it can reach more than 1000 ppm at the exit of a tunnel on a road. The concentration chosen for the “accelerated” CO₂ capture experiments was 10,000 ppm, which is 10 times more concentrated than at the exit of a tunnel for example.

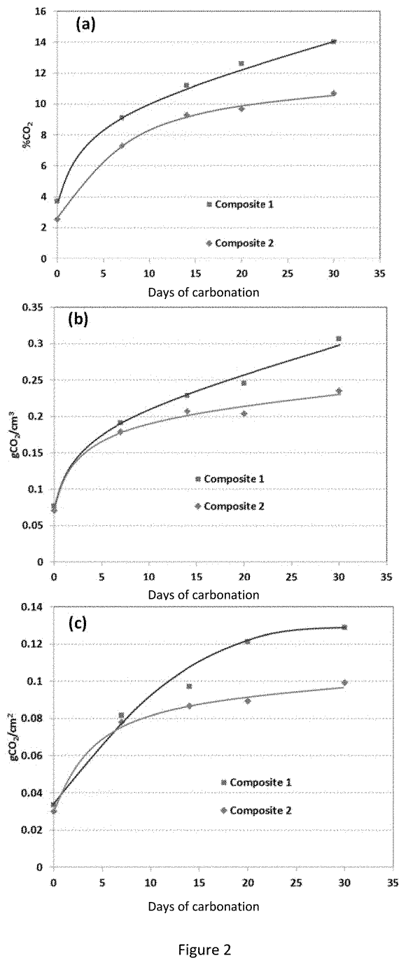

FIG. 2 shows the amount of CO₂ captured for both composites 1 and 2 (prepared in Examples 1 and 2), as a function of the number of days in the chamber (carbonation days). In this example, the composite material according to the invention contains 60% by volume of aggregates of recycled concrete (with an average diameter of about 6 mm) and 40% by volume of porous carbon particles (with an average diameter of about 6 mm, obtained from biomass and with a specific surface area of 1300 m²/g) in a cement matrix (cement type CEM1).

To show the synergistic effect of composite 1, it is compared with composite 2 (not comprising porous carbon). The results presented in FIG. 2 show that composite 1 has a significantly higher CO₂ adsorption capacity than composite 2.

In particular, after 30 days of exposure to CO₂, the composite 1 presents:
an adsorption capacity (for an equivalent mass of material) greater than 20% (FIG. 2a),
an adsorption capacity (for an equivalent volume of material) greater than 30% (FIG. 2b), and
an adsorption capacity (for an equivalent surface of material exposed to the pollutant gas) greater than 30% (FIG. 2c).

Thus, a synergistic effect on the absorption of pollutant gases is observed between the composite 1 according to the invention and the composite 2 out of the invention. Surprisingly, the CO₂ adsorption capacity of composite 1 is not only significantly higher (by mass, by volume and by exposed surface) but also faster than that of composite 2.

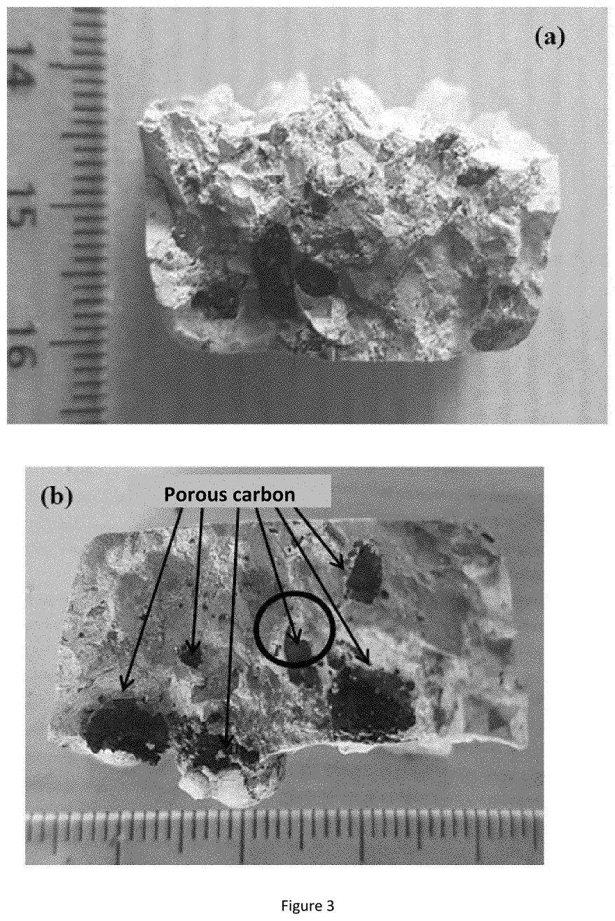

In FIG. 3 (composite 2, FIG. 3a and composite 1, FIG. 3b) after 20 days under an atmosphere of 10,000 ppm CO₂, carbonation of the cementitious material (uncolored areas) around the carbon particles is observed for composite 1. This indicates that in the case of composite 1, the CO₂ gas which is not adsorbed in the carbon porosity diffuses around the particle and reaches the whole matrix more quickly than in the case of composite 2.

1. A fresh composite comprising at least one concrete aggregate having a porosity greater than or equal to 12%, porous carbon having a micropore volume greater than or equal to 0.2 cm³/g and a macropore volume greater than or equal to 0.2 cm³/g, a binder and optionally water.

2. The fresh composite of claim 1, wherein the volume ratio of concrete aggregate/porous carbon is in the range of 30:70 to 80:20.

3. The fresh composite of claim 1, wherein the ratio of concrete aggregate outer surface/porous carbon outer surface is in the range of 0.5 to 1.5.

4. The fresh composite of claim 1, wherein the mass ratio of concrete aggregate/binder is in the range of 0.6 to 1.

5. The fresh composite of claim 1, wherein the porous carbon/binder weight ratio is in the range of 0.03 to 0.1.

6. The fresh composite of claim 1, wherein the porous carbon consists essentially of carbon and has a specific surface area ranging from 500 to 3000 m²/g.

7. The fresh composite of claim 1, wherein the porous carbon aggregate is functionalized.

8. The fresh composite of claim 1, wherein the porous carbon aggregate has an average diameter ranging from 1 to 20 mm.

9. The fresh composite of claim 1, wherein the concrete aggregate consists essentially of recycled concrete.

10. The fresh composite of claim 1, wherein the concrete aggregate has an average diameter ranging from 1 to 50 mm.

11. The fresh composite of claim 1, wherein the water/cement ratio has a value ranging from 0.3 to 0.6.

12. The fresh composite of claim 1, wherein the binder comprises at least one binder selected from CEM I, CEM II, CEM III, CEM IV or CEM V cements, polymeric type binders, resins or carbon-based binders, or mixtures thereof.

13. The fresh composite of claim 1, further comprising at least one adjuvant and/or additive.

14. A composite comprising at least one concrete aggregate having a porosity greater than or equal to 12%, porous carbon having a micropore volume greater than or equal to 0.2 cm³/g and a macropore volume greater than or equal to 0.2 cm³/g, a binder and optionally water.

15. A method for sanitizing air comprising placing the fresh composite of claim 1 into an indoor or outdoor environment.

16. A method for manufacturing the composite of claim 14, comprising the steps of:
a) mixing at least one concrete aggregate having a porosity greater than or equal to 12%, porous carbon having a micropore volume greater than or equal to 0.2 cm³/g and a macropore volume greater than or equal to 0.2 cm³/g, a binder, optionally water and optionally additives, adjuvants and/or aggregates, and obtaining a fresh composite;
b) molding and curing the fresh composite obtained in step a); and
c) demolding.

17. The fresh composite of claim 7, wherein the porous carbon aggregate is functionalized on the surface with at least one heteroatom selected from O, N, S or P.

18. The fresh composite of claim 8, wherein the porous carbon aggregate has an average diameter ranging from 5 to 10 mm.

19. The fresh composite of claim 10, wherein the concrete aggregate has an average diameter ranging from 5 to 10 mm.

20. The method of claim 15, wherein the fresh composite absorbs CO₂, SO₂, NOx and/or VOCs.
